(19) United States
(12) Patent Application Publication
(10) Pub. No.: US 2025/0262435 A1
(43) Pub. Date: Aug. 21, 2025

(54) COCHLEAR IMPLANT SYSTEM WITH IMPLANTABLE BODY SENSORS

(71) Applicant: Envoy Medical Corporation, White Bear Lake, MN (US)

(72) Inventors: Brent Thomas Lucas, North Oaks, MN (US); Thomas B. Hoegh, Edina, MN (US)

(21) Appl. No.: 19/058,941

(22) Filed: Feb. 20, 2025

Related U.S. Application Data
(60) Provisional application No. 63/556,231, filed on Feb. 21, 2024.

(51) Int. Cl. A61N 1/36; A61N 1/372; A61N 1/378

(52) U.S. Cl. CPC A61N 1/36038; A61N 1/36139; A61N 1/3614; A61N 1/37258; A61N 1/3787

(57) ABSTRACT

Implantable systems can include an implantable body sensor configured to measure a first body parameter and generate a first body parameter signal representative of the first body parameter and a cochlear implant system in communication with the implantable body sensor. The cochlear implant system can be configured to provide electrical stimulation to cochlear tissue in response to one or more input signals and receive the first body parameter signal from the implantable body sensor. The cochlear implant system can be configured to provide electrical stimulation to cochlear tissue based on the received first body parameter signal and representative of the first body parameter such that a wearer perceives a representation of the first body parameter. The cochlear implant system can be configured to output body parameter information representative of the first body parameter to an external device.

### CROSS-REFERENCE

This application claims priority to U.S. Provisional Patent Application 63/556,231, filed Feb. 21, 2024, the entire contents of which is incorporated herein by reference.

### BACKGROUND

A cochlear implant is an electronic device that may be at least partially implanted surgically into the cochlea, the hearing organ of the inner ear, to provide improved hearing to a patient. Cochlear implants may include components that are worn externally by the patient and components that are implanted internally in the patient. Implant system can receive acoustic stimuli and output electrical stimuli to a patient in response thereto to effectively enable a patient to hear.

### SUMMARY

Some aspects of the disclosure include cochlear implant systems and implantable body sensors. Implantable body sensor can be configured to measure a body parameter and generate a body parameter signal representative of the body parameter.

The cochlear implant system can be configured to provide electrical stimulation to cochlear tissue in response to one or more input signals and to receive the body parameter signal from the implantable body sensor. In some examples, the cochlear implant system is configured to provide electrical stimulation to the cochlear tissue based on a received body parameter signal and representative of the body parameter such that a wearer perceives a representation of the body parameter.

A cochlear implant system can include a cochlear electrode, a stimulator in electrical communication with the cochlear electrode, an input source, a signal processor, and an implantable battery and/or communication module. The signal processor can be configured to receive an input signal from the input source and output a stimulation signal to the stimulator based on the received input signal and a transfer function of the signal processor. The implantable battery and/or communication module can be configured to provide electrical power to the signal processor.

In some embodiments, the implantable battery and/or communication module is configured to receive a body parameter signal from the implantable body sensor. The implantable battery and/or communication module can be configured to output body parameter information based on the received body parameter signal to the signal processor. The signal processor can be configured update the transfer function based on the received body parameter information.

In some examples, cochlear implant system is configured to output body parameter information representative of the body parameter to an external device, such as via Bluetooth communication between the implantable battery and/or communication module and the external device.

Sensors configured to measure various body parameters are possible, including a heart rate, body temperature, an electrocardiogram, electroencephalogram (EEG), blood oxygen levels, glucose levels, inspiratory effort levels, inspiratory onset, inspiratory offset, body orientation, body impedance measurements (e.g., real and/or imaginary body impedance components), wearer activity level, or sleep stage information. Combinations of various body sensors are possible.

### BRIEF DESCRIPTION OF THE DRAWINGS

FIG. 1 shows a schematic illustration of a fully implantable cochlear implant system.

FIG. 2 illustrates an embodiment of a fully-implantable cochlear implant.

FIG. 3 illustrates an embodiment of an exemplary middle ear sensor for use in conjunction with anatomical features of a patient.

FIG. 4 is a schematic diagram illustrating an exemplary implantable system including an acoustic stimulator.

FIGS. 5A and 5B show high-level schematic diagrams illustrating example communication configurations between an implantable battery and/or communication module, a signal processor, and a stimulator.

FIGS. 6A-6C show example communication arrangements between an example body sensor, signal processor, and implantable battery and/or communication module.

FIG. 7 is a schematic diagram of an example cochlear implant system including an exemplary implantable cochlear implant system and an external device.

FIG. 8 is a process flow diagram showing an example process for monitoring a body parameter.

### DETAILED DESCRIPTION

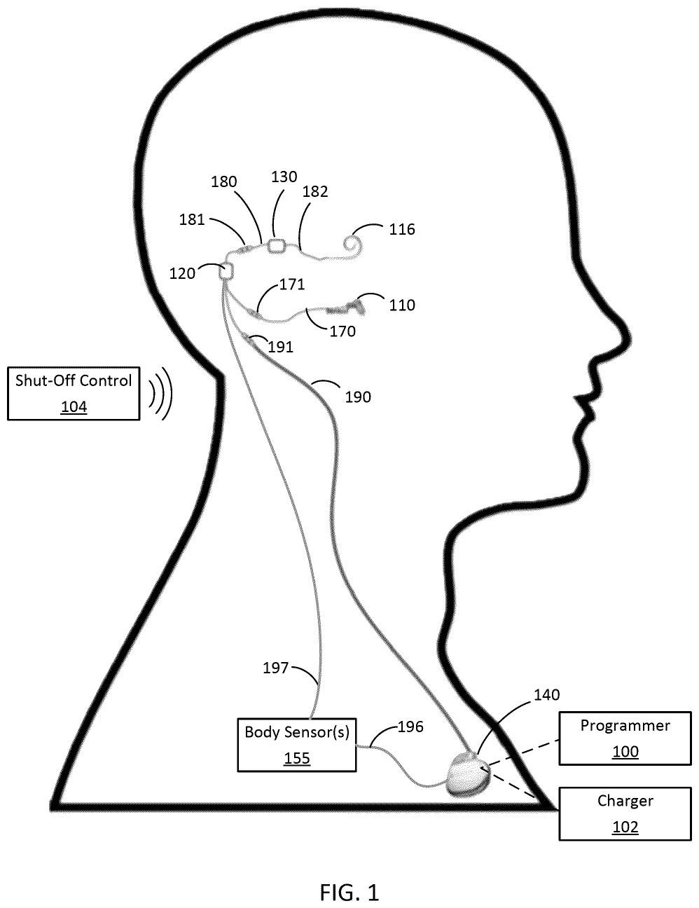

FIG. 1 shows a schematic illustration of a fully implantable cochlear implant system. The system of FIG. 1 includes a middle ear sensor 110 in communication with a signal processor 120. The middle ear sensor 110 can be configured to detect incoming sound waves, for example, using the ear structure of a patient. The signal processor 120 can be configured to receive a signal from the middle ear sensor 110 and produce an output signal based thereon. For example, the signal processor 120 can be programmed with instructions to output a certain signal based on a received signal. In some embodiments, the output of the signal processor 120 can be calculated using an equation based on received input signals. Alternatively, in some embodiments, the output of the signal processor 120 can be based on a lookup table or other programmed (e.g., in memory) correspondence between the input signal from the middle ear sensor 110 and the output signal. While not necessarily based explicitly on a function, the relationship between the input to the signal processor 120 (e.g., from the middle ear sensor 110) and the output of the signal processor 120 is referred to as the transfer function of the signal processor 120.

In various examples, the signal processor 120 can comprise any variety of components, for example, digital and/or analog processing components. In some embodiments, signal processor 120 comprises a digital signal processor, one or more microprocessors, microcontrollers, application specific integrated circuits (ASICs) or the like. Supporting circuitry for one or more such components can also be included as a part of the signal processor. In some embodiments, the signal processor can include or otherwise communicate with a memory containing programming for operating one or more components. Additionally or alternatively, in some embodiments, the signal processor can include one or more additional components. For example, in some embodiments, signal processor can include an embedded microphone or other sensor configured to detect incoming sound waves.

The system of FIG. 1 further includes a cochlear electrode 116 implanted into the cochlear tissues of a patient. The cochlear electrode 116 is in electrical communication with an electrical stimulator 130, which can be configured to provide electrical signals to the cochlear electrode 116 in response to input signals received by the electrical stimulator 130. In some examples, the cochlear electrode 116 is fixedly attached to the electrical stimulator 130. In other examples, the cochlear electrode 116 is removably attached to the electrical stimulator 130. As shown, the electrical stimulator 130 is in communication with the signal processor 120. In some embodiments, the electrical stimulator 130 provides electrical signals to the cochlear electrode 116 based on output signals from the signal processor 120.

In various embodiments, the cochlear electrode 116 can include any number of contact electrodes in electrical contact with different parts of the cochlear tissue. In such embodiments, the electrical stimulator 130 can be configured to provide electrical signals to any number of such contact electrodes to stimulate the cochlear tissue. For example, in some embodiments, the electrical stimulator 130 is configured to activate different contact electrodes or combinations of contact electrodes of the cochlear electrode 116 in response to different input signals received from the signal processor 120. This can help the patient differentiate between different input signals. One or more return electrodes can be included as part of the stimulator 130, signal processor 120, or cochlear electrode 116 to provide a return path for the electrical stimulation.

During exemplary operation, the middle ear sensor 110 detects audio signals, for example, using features of the patient's ear anatomy as described elsewhere herein and in U.S. Patent Publication No. 2013/0018216, which is hereby incorporated by reference in its entirety. The signal processor 120 can receive such signals from the middle ear sensor 110 and produce an output to the electrical stimulator 130 based on the transfer function of the signal processor 120. The electrical stimulator 130 can then stimulate one or more contact electrodes of the cochlear electrode 116 based on the received signals from the signal processor 120.

In some embodiments, a system can include one or more body sensors 155. Body sensor(s) can be configured to sense one or more body parameters, such as a measurable physiological parameter associated with the wearer of the cochlear implant system, and output a body parameter signal representative of the body parameter. Example body sensors can be configured to measure body parameters such as heart rate, body temperature, an electrocardiogram, electroencephalogram (EEG), blood oxygen levels, glucose levels, inspiratory effort levels, body orientation, body impedance measurements, wearer activity level, sleep stages, or other measurable physiological parameters. In some embodiments, a body sensor can be used to detect specific events associated with a particular body location. For example, an accelerometer in a chest area of a wearer can provide information regarding external impacts on the chest.

In some examples, a body sensor can include an electrode, for example, to measure one or more body parameters, such as one or more electrical parameters. In some examples, body sensor(s) 155 are fully implanted or implantable into the wearer. In other examples, body sensor(s) can be used externally. In some embodiments, one or more body sensors can operate without electrical power supplied thereto. For example, some body sensors can operate without electricity. Other examples can include one or more power sources (e.g., one or more batteries, such as rechargeable or replaceable batteries) such that no external power is required for operation. Additionally or alternatively, a body sensor can receive power from one or more implanted components, such as implantable battery and/or communication module 140.

In some examples, one or more body sensors 155 can be configured to communicate with one or more implanted system components, such as an implantable battery and/or communication module 140 and/or signal processor 120. In the illustrated embodiment, one or more body sensors 155 are shown as being in communication with implantable battery and/or communication module 140 via a lead 196, and with signal processor 120 via lead 197. In some such embodiments, body sensor(s) 155 can receive electrical power for operation from implantable battery and/or communication module 140 via lead 196, and can provide a body parameter signal to signal processor 120 via lead 197.

In some examples, one of leads 197 and 196 can be eliminated. For example, as described elsewhere herein, in some embodiments, body sensor(s) 155 can be in communication with signal processor 120 via a lead (e.g., lead 197) and can receive power from the signal processor 120 via the lead, for example, by directing power received at the signal processor 120 from the implantable battery and/or communication module 140 through to the body sensor(s) 155. In other examples, body sensor(s) 155 can be in communication with an implantable battery and/or communication module 140 via a lead (e.g., lead 196) and receive power therefrom. The body sensor(s) 155 can output a body parameter signal to the implantable battery and/or communication module 140, which can relay the body parameter signal (or a signal representative thereof) to the signal processor 120.

In some examples including multiple body sensors 155, different body sensors can be in communication with different ones of the signal processor 120 and implantable battery and/or communication module 140, such as depending on convenience of location or the like. For instance, in an embodiment in which implantable battery and/or communication module 140 is implanted in the pectoral region of a wearer, a body sensor implanted in or near the pectoral region can be configured to communicate with implantable battery and/or communication module via a lead (e.g., 196). However, in some such instances, the body sensor is not placed in direct communication with a signal processor positioned in the head of the wearer via a lead (e.g., lead 197). Instead, in some such examples, information from the body sensor can be communicated to the implantable battery and/or communication module, and then relayed to the signal processor (e.g., via lead 190). Similarly, a body sensor positioned in or near the head of the wearer could be configured to communicate directly with signal processor 120, such as via lead 197, without being directly connected to implantable battery and/or communication module 140 via lead 196. In some such examples, such a body sensor can receive any power necessary for its operation from signal processor 120 via lead 197.

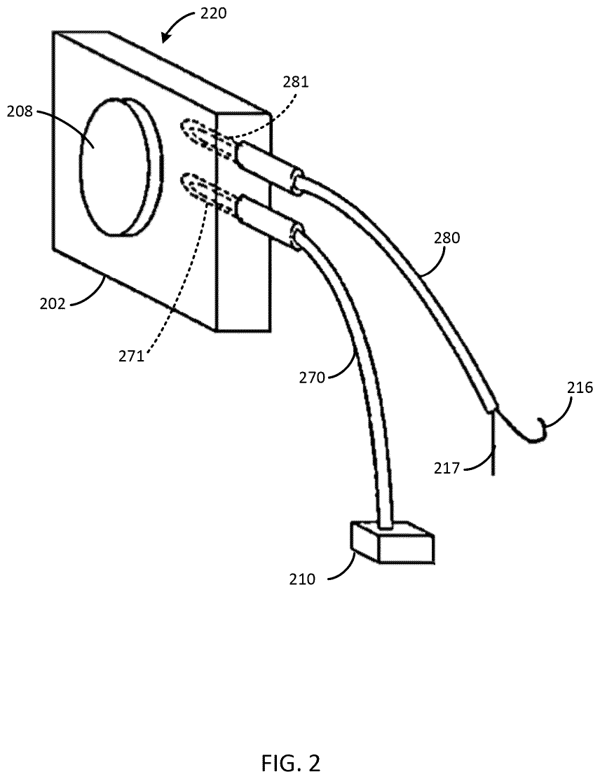

Referring to FIG. 2, an embodiment of a fully-implantable cochlear implant is shown. The device in this embodiment includes a processor 220 (e.g., signal processor), a sensor 210, a first lead 270 connecting the sensor 210 to the processor 220, and a combination lead 280 attached to the processor 220, wherein combination lead 280 contains both a ground electrode 217 and a cochlear electrode 216. The illustrated processor 220 includes a housing 202, a coil 208, first female receptacle 271 and second female receptacle 281 for insertion of the leads 270 and 280, respectively.

In some embodiments, coil 208 can receive power and/or data from an external device, for instance, including a transmission coil (not shown). Some such examples are described in U.S. Patent Publication No. 2013/0018216, which is incorporated by reference. In other examples, processor 220 is configured to receive power and/or data from other sources, such as an implantable battery and/or communication module as shown in FIG. 1. Such battery and/or communication module can be implanted, for example, into the pectoral region of the patient in order to provide adequate room for larger equipment (e.g., a relatively large battery) for prolonged operation (e.g., longer battery life). Additionally, in the event a battery needs eventual replacement, a replacement procedure in the patient's pectoral region can be performed several times without certain vascularization issues that can arise near the location of the cochlear implant. For example, in some cases, repeated procedures (e.g., battery replacement) near the cochlear implant can result in a decreased ability for the skin in the region to heal after a procedure. Placing a replaceable component such as a battery in the pectoral region can facilitate replacement procedures with reduced risk for such issues.

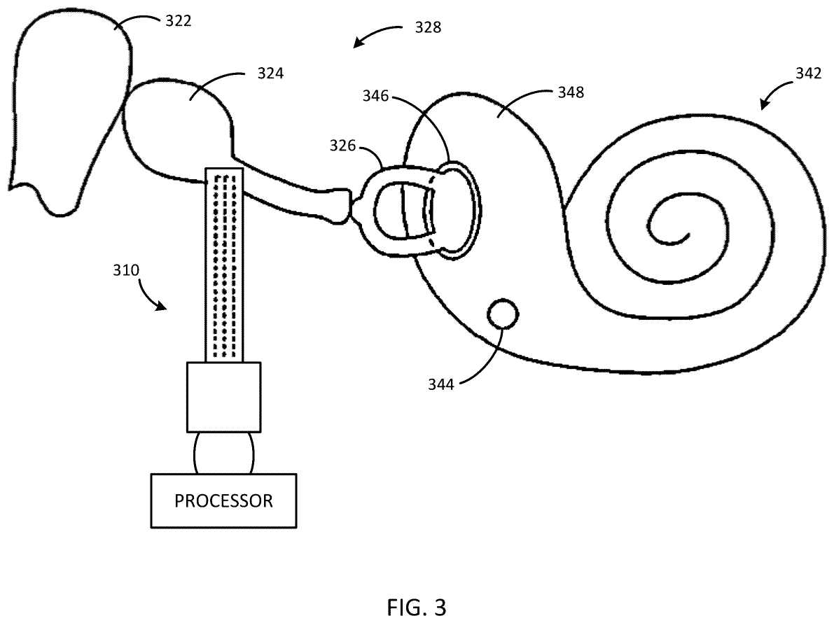

FIG. 3 illustrates embodiments of an exemplary middle ear sensor for use in conjunction with anatomical features of a patient. Referring to FIG. 3, an embodiment of the sensor 310 of a fully-implantable cochlear implant is shown. Also shown are portions of the subject's anatomy, which includes, if the subject is anatomically normal, at least the malleus 322, incus 324, and stapes 326 of the middle ear 328, and the cochlea 348, oval window 346, and round window 344 of the inner ear 342. Here, the sensor 310 is touching the incus 324. The sensor 310 can include a sensor such as described in U.S. Patent Publication No. 2013/0018216, which is incorporated by reference. Further, although not shown in a drawing, the sensor 310 may be in operative contact with the tympanic membrane or the stapes, or any combination of the tympanic membrane, malleus 322, incus 324, or stapes 326.

FIG. 3 illustrates an exemplary middle ear sensor for use with systems described herein. However, other middle ear sensors can be used, such as sensors using microphones or other sensors capable of receiving an input corresponding to detected sound and outputting a corresponding signal to the signal processor. Additionally or alternatively, systems can include other sensors configured to output a signal representative of sound received at or near a user's ear, such as a microphone or other acoustic pickup located in the user's outer ear or implanted under the user's skin. Such devices may function as an input source, for example, to the signal processor such that the signal processor receives an input signal from the input source and generates and output one or more stimulation signals according to the received input signal and the signal processor transfer function. Additionally or alternatively, systems can include other types of sensors, such as inner ear sensors. Some example configurations of such systems and other sensor arrangements are described in PCT patent application No. PCT/US20/19166, filed Feb. 21, 2020, which is assigned to the assignee of the instant application and is incorporated by reference.

Referring back to FIG. 1, the signal processor 120 is shown as being in communication with the middle ear sensor 110, the electrical stimulator 130, and the implantable battery and/or communication module 140. As described elsewhere herein, the signal processor 120 can receive input signals from the middle ear sensor 110 and/or other input source(s) and output signals to the electrical stimulator 130 for stimulating the cochlear electrode 116. The signal processor 120 can receive data (e.g., processing data establishing or updating the transfer function of the signal processor 120) and/or power from the implantable battery and/or communication module 140.

In some embodiments, the implantable battery and/or communication module 140 can communicate with one or more external components, such as a programmer 100 and/or a battery charger 102. The battery charger 102 can wirelessly charge the battery in the implantable battery and/or communication module 140 when brought into proximity with the implantable battery and/or communication module 140 in the pectoral region of the patient. Such charging can be accomplished, for example, using inductive charging. The programmer 100 can be configured to wirelessly communicate with the implantable battery and/or communication module 140 via any appropriate wireless communication technology, such as Bluetooth, Wi-Fi, and the like. In some examples, the programmer 100 can be used to update the system firmware and/or software. In an exemplary operation, the programmer 100 can be used to communicate an updated signal processor 120 transfer function to the implantable battery and/or communication module 140. In various embodiments, the programmer 100 and charger 102 can be separate devices or can be integrated into a single device.

In the illustrated example of FIG. 1, the signal processor 120 is connected to the middle ear sensor 110 via lead 170. In some embodiments, lead 170 can provide communication between the signal processor 120 and the middle ear sensor 110. In some embodiments, lead 170 can include a plurality of isolated conductors providing a plurality of communication channels between the middle ear sensor 110 and the signal processor 120. The lead 170 can include a coating such as an electrically insulating sheath to minimize any conduction of electrical signals to the body of the patient. In various embodiments, one or more communication leads can be detachable such that communication between two components can be disconnected in order to electrically and/or mechanically separate such components. For instance, in some embodiments, lead 170 includes a detachable connector 171. Detachable connector 171 can facilitate decoupling of the signal processor 120 and middle ear sensor 110. Example detachable connectors are described in PCT patent application No. PCT/US20/19166, which is incorporated by reference. For example, with reference to FIG. 1, in some embodiments, lead 170 can include a first lead extending from the middle ear sensor 110 having one of a male or a female connector and a second lead extending from the signal processor 120 having the other of the male or female connector. The first and second leads can be connected at detachable connector 171 in order to facilitate communication between the middle ear sensor 110 and the signal processor 120.

In other examples, a part of the detachable connector 171 can be integrated into one of the middle ear sensor 110 and the signal processor 120. For example, in an exemplary embodiment, the signal processor 120 can include a female connector integrated into a housing of the signal processor 120. Lead 170 can extend fully from the middle ear sensor 110 and terminate at a corresponding male connector for inserting into the female connector of the signal processor 120. In still further embodiments, a lead (e.g., 170) can include connectors on each end configured to detachably connect with connectors integrated into each of the components in communication. For example, lead 170 can include two male connectors, two female connectors, or one male and one female connector for detachably connecting with corresponding connectors integral to the middle ear sensor 110 and the signal processor 120. Thus, lead 170 may include two or more detachable connectors.

Similar communication configurations can be established for detachable connector 181 of lead 180 facilitating communication between the signal processor 120 and the stimulator 130 and for detachable connector 191 of lead 190 facilitating communication between the signal processor 120 and the implantable battery and/or communication module 140. Leads (170, 180, 190) can include pairs of leads having corresponding connectors extending from each piece of communicating equipment, or connectors can be built in to any one or more communicating components.

In such configurations, each of the electrical stimulator 130, signal processor 120, middle ear sensor 110, and battery and/or communication module can each be enclosed in a housing, such as a hermetically sealed housing comprising biocompatible materials. Such components can include feedthroughs providing communication to internal components enclosed in the housing. Feedthroughs can provide electrical communication to the component via leads extending from the housing and/or connectors integrated into the components.

In a module configuration such as that shown in FIG. 1, various components can be accessed (e.g., for upgrades, repair, replacement, etc.) individually from other components. For example, as signal processor 120 technology improves (e.g., improvements in size, processing speed, power consumption, etc.), the signal processor 120 implanted as part of the system can be removed and replaced independently of other components. In an exemplary procedure, an implanted signal processor 120 can be disconnected from the electrical stimulator 130 by disconnecting detachable connector 181, from the middle ear sensor 110 by disconnecting detachable connector 171, and from the implantable battery and/or communication module 140 by disconnecting detachable connector 191. Thus, the signal processor 120 can be removed from the patient while other components such as the electrical stimulator 130, cochlear electrode 116, middle ear sensor 110, and battery and/or communication module can remain in place in the patient.

After the old signal processor is removed, a new signal processor can be connected to the electrical stimulator 130, middle ear sensor 110, and implantable battery and/or communication module 140 via detachable connectors 181, 171, and 191, respectively. Thus, the signal processor (e.g., 120) can be replaced, repaired, upgraded, or any combination thereof, without affecting the other system components. This can reduce, among other things, the risk, complexity, duration, and recovery time of such a procedure. In particular, the cochlear electrode 116 can be left in place in the patient's cochlea while other system components can be adjusted, reducing trauma to the patient's cochlear tissue.

Such modularity of system components can be particularly advantageous when replacing a signal processor 120, such as described above. Processor technology continues to improve and will likely continue to markedly improve in the future, making the signal processor 120 a likely candidate for significant upgrades and/or replacement during the patient's lifetime. Additionally, in embodiments such as the embodiment shown in FIG. 1, the signal processor 120 communicates with many system components. For example, as shown, the signal processor 120 is in communication with each of the electrical stimulator 130, the middle ear sensor 110, and the implantable battery and/or communication module 140. Detachably connecting such components with the signal processor 120 (e.g., via detachable connectors 181, 171, and 191) enables replacement of the signal processor 120 without disturbing any other components. Thus, in the event of an available signal processor 120 upgrade and/or a failure of the signal processor 120, the signal processor 120 can be disconnected from other system components and removed.

While many advantages exist for a replaceable signal processor 120, the modularity of other system components can be similarly advantageous, for example, for upgrading any system component. Similarly, if a system component (e.g., the middle ear sensor 110) should fail, the component can be disconnected from the rest of the system (e.g., via detachable connector 171) and replaced without disturbing the remaining system components. In another example, even a rechargeable battery included in the implantable battery and/or communication module 140 may eventually wear out and need replacement. The implantable battery and/or communication module 140 can be replaced or accessed (e.g., for replacing the battery) without disturbing other system components. Further, as discussed elsewhere herein, when the implantable battery and/or communication module 140 is implanted in the pectoral region of the patient, such as in the illustrated example, such a procedure can leave the patient's head untouched, eliminating unnecessarily frequent access beneath the skin.

In some examples, one or more body sensors 155 can be detachably connected to one or more system components with which the body sensor(s) communicate. For example, in some embodiments in which one or more body sensors 155 communicate with signal processor 120 via lead 197, the body sensor(s) 155 can be detachable from signal processor 120. For instance, in some such embodiments, lead 197 coupling body sensor(s) 155 and signal processor 120 comprises a detachable connector and/or is detachable from one or both of body sensor(s) 155 and signal processor 120. Additionally or alternatively, body sensor(s) 155 can be detachably couplable to implantable battery and/or communication module 140, such as by a lead 196 comprising a detachable connector or otherwise being detachable from body sensor(s) 155 and/or implantable battery and/or communication module 140. In various examples, being able to detach one or more body sensors from system components can facilitate removal or replacement (e.g., for repair or upgrade) of the body sensor(s) and/or components that communicate therewith.

While various components are described herein as being detachable, in various embodiments, one or more components configured to communicate with one another can be integrated into a single housing. For example, in some embodiments, signal processor 120 can be integrally formed with the stimulator 130 and cochlear electrode 116. For example, in an exemplary embodiment, processing and stimulation circuitry of a signal processor 120 and stimulator 130 can be integrally formed as a single unit in a housing coupled to a cochlear electrode. A return electrode can be coupled to the housing from which the cochlear electrode extends. Cochlear electrode and the signal processor/stimulator can be implanted during an initial procedure and operate as a single unit.

In some embodiments, while the integral signal processor/stimulator/cochlear electrode component does not get removed from a patient due to potential damage to the cochlear tissue into which the cochlear electrode is implanted, system upgrades are still possible. For example, in some embodiments, a modular signal processor may be implanted alongside the integral signal processor/stimulator component and communicate therewith. In some such examples, the integral signal processor may include a built-in bypass to allow a later-implanted signal processor to interface directly with the stimulator. Additionally or alternatively, the modular signal processor can communicate with the integral signal processor, which may be programmed with a unity transfer function. Thus, in some such embodiments, signals from the modular signal processor may be essentially passed through the integral signal processor unchanged so that the modular signal processor effectively controls action of the integral stimulator. Thus, in various embodiments, hardware and/or software solutions exist for upgrading an integrally attached signal processor that may be difficult or dangerous to remove.

While often described herein as using an electrical stimulator to stimulate the patient's cochlear tissue via a cochlear electrode, in some examples, the system can additionally or alternatively include an acoustic stimulator. An acoustic stimulator can include, for example, a transducer (e.g., a piezoelectric transducer) configured to provide mechanical stimulation to the patient's ear structure. In an exemplary embodiment, the acoustic stimulator can be configured to stimulate one or more portions of the patient's ossicular chain via amplified vibrations. Acoustic stimulators can include any appropriate acoustic stimulators, such as those found in the ESTEEM™ implant (Envoy Medical Corp., St. Paul, Minn.) or as described in U.S. Pat. Nos. 4,729,366, 4,850,962, and 7,524,278, and U.S. Patent Publication No. 2010/0042183, each of which is incorporated herein by reference in its entirety.

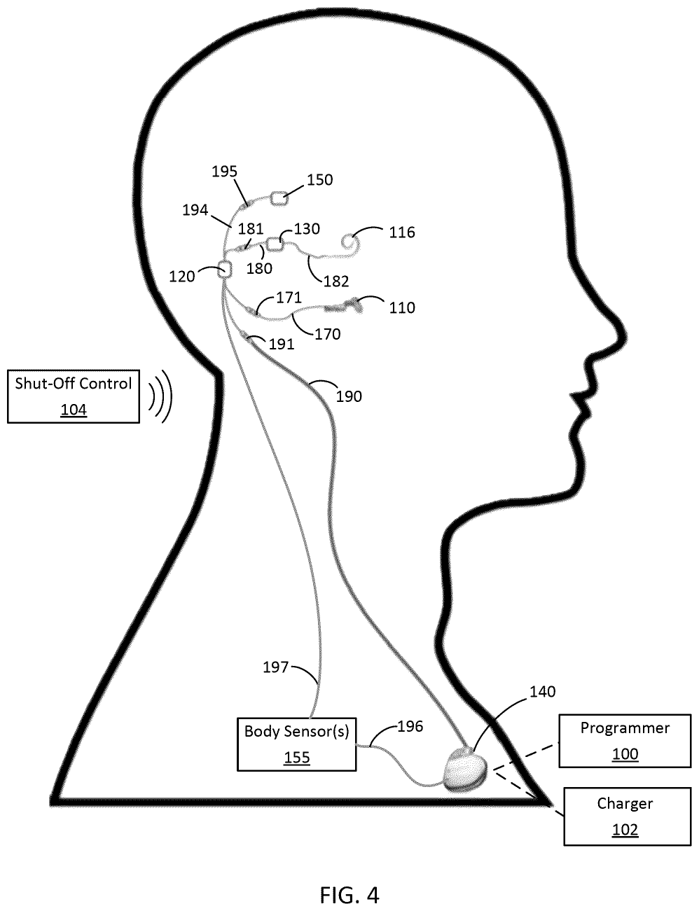

FIG. 4 is a schematic diagram illustrating an exemplary implantable system including an acoustic stimulator. The acoustic stimulator can be implanted proximate the patient's ossicular chain and can be in communication with a signal processor via lead 194 and detachable connector 195. The signal processor can behave as described elsewhere herein and can be configured to cause acoustic stimulation of the ossicular chain via the acoustic stimulator in in response to input signals from the middle ear sensor according to a transfer function of the signal processor.

The acoustic stimulator of FIG. 4 can be used similarly to the electrical stimulator as described elsewhere herein. For instance, an acoustic stimulator can be mechanically coupled to a patient's ossicular chain upon implanting the system and coupled to the signal processor via lead 194 and detachable connector 195. Similarly to systems described elsewhere herein with respect to the electrical stimulator, if the signal processor requires replacement or repair, the signal processor can be disconnected from the acoustic stimulator (via detachable connector 195) so that the signal processor can be removed without disturbing the acoustic stimulator.

In general, systems incorporating an acoustic stimulator such as shown in FIG. 4 can operate in the same way as systems described elsewhere herein employing an electrical stimulator and cochlear electrode only substituting or supplementing electrical stimulation with acoustic stimulation.

Some systems can include a hybrid system comprising both an electrical stimulator and an acoustic stimulator in communication with the signal processor. In some such examples, the signal processor can be configured to stimulate electrically and/or acoustically according to the transfer function of the signal processor. In some examples, the type of stimulation used can depend on the input signal received by the signal processor. For instance, in an exemplary embodiment, the frequency content of the input signal to the signal processor can dictate the type of stimulation. In some cases, frequencies below a threshold frequency could be represented using one of electrical and acoustic stimulation while frequencies above the threshold frequency could be represented using the other of electrical and acoustic stimulation. Such a threshold frequency could be adjustable based on the hearing profile of the patient. Using a limited range of frequencies can reduce the number of frequency domains, and thus the number of contact electrodes, on the cochlear electrode. In other examples, rather than a single threshold frequency defining which frequencies are stimulated electrically and acoustically, various frequencies can be stimulated both electrically and acoustically. In some such examples, the relative amount of electrical and acoustic stimulation can be frequency-dependent. As described elsewhere herein, the signal processor transfer function can be updated to meet the needs of the patient, including the electrical and acoustic stimulation profiles.

Additionally or alternatively, while many examples show a middle ear sensor being in communication with an implanted signal processor, in various embodiments, one or more additional or alternative input sources can be included. For instance, in some embodiments, a microphone can be implanted under a user's skin and can be placed in communication with the signal processor (e.g., via a detachable connector such as 171). The signal processor can receive input signals from the implanted microphone and provide signals to the stimulator based on the received input signal and the signal processor transfer function. Additionally or alternatively, systems can include a middle ear sensor as an input source, wherein the middle ear sensor is configured to detect stimuli (e.g., pressure signals) from the wearer's inner ear (e.g., within the cochlear tissue).

With further reference to FIGS. 1 and 4, in some examples, a system can include a shut-off controller 104, which can be configured to wirelessly stop an electrical stimulator 130 from stimulating the patient's cochlear tissue and/or an acoustic stimulator 150 from stimulating the patient's ossicular chain. For example, if the system is malfunctioning or an uncomfortably loud input sound causes an undesirable level of stimulation, the user may use the shut-off controller 104 to cease stimulation from the stimulator 130. The shut-off controller 104 can be embodied in a variety of ways. For example, in some embodiments, the shut-off controller 104 can be integrated into other external components, such as the programmer 100. In some such examples, the programmer 100 includes a user interface by which a user can select an emergency shut-off feature to cease stimulation. Additionally or alternatively, the shut-off controller 104 can be embodied as a separate component. This can be useful in situations in which the patient may not have immediate access to the programmer 100. For example, the shut-off controller 104 can be implemented as a wearable component that the patient can wear at all or most times, such as a ring, bracelet, necklace, or the like.

The shut-off controller 104 can communicate with the system in order to stop stimulation in a variety of ways. In some examples, the shut-off controller 104 comprises a magnet that is detectable by a sensor (e.g., a Hall-Effect sensor) implanted in the patient, such as in the processor and/or the implantable battery and/or communication module 140. In some such embodiments, when the magnet is brought sufficiently close to the sensor, the system can stop stimulation of the cochlear tissue or ossicular chain.

After the shut-off controller 104 is used to disable stimulation, stimulation can be re-enabled in one or more of a variety of ways. For example, in some embodiments, stimulation is re-enabled after a predetermined amount of time after it had been disabled. In other examples, the shut-off controller 104 can be used to re-enable stimulation. In some such examples, the patient brings the shut-off controller 104 within a first distance of a sensor (e.g., a magnetic sensor) to disable stimulation, and then removes the shut-off controller 104. Subsequently, once the patient brings the shut-off controller 104 within a second distance of the sensor, stimulation can be re-enabled. In various embodiments, the first distance can be less than the second distance, equal to the second distance, or greater than the second distance. In still further embodiments, another device such as a separate turn-on controller (not shown) or the programmer 100 can be used to re-enable stimulation. Any combination of such re-enabling of stimulation can be used, such as alternatively using either the programmer 100 or the shut-off controller 104 to enable stimulation or combining a minimum “off” time before any other methods can be used to re-enable stimulation.

In some embodiments, rather than entirely disable stimulation, other actions can be taken, such as reducing the magnitude of stimulation. For example, in some embodiments, the shut-off sensor can be used to reduce the signal output by a predetermined amount (e.g., absolute amount, percentage, etc.). In other examples, the shut-off sensor can affect the transfer function of the signal processor to reduce the magnitude of stimulation in a customized way, such as according to frequency or other parameter of an input signal (e.g., from the middle ear sensor).

In some examples, implantable battery and/or communication module can be used to provide power and/or data (e.g., processing instructions) to various system components via lead 190. Different challenges exist for communicating electrical signals through a patient's body. For example, safety standards can limit the amount of current that can safely flow through a patient's body (particularly DC current). Additionally, the patient's body can act as an undesired signal path from component to component (e.g., via contact with the housing or “can” of each component).

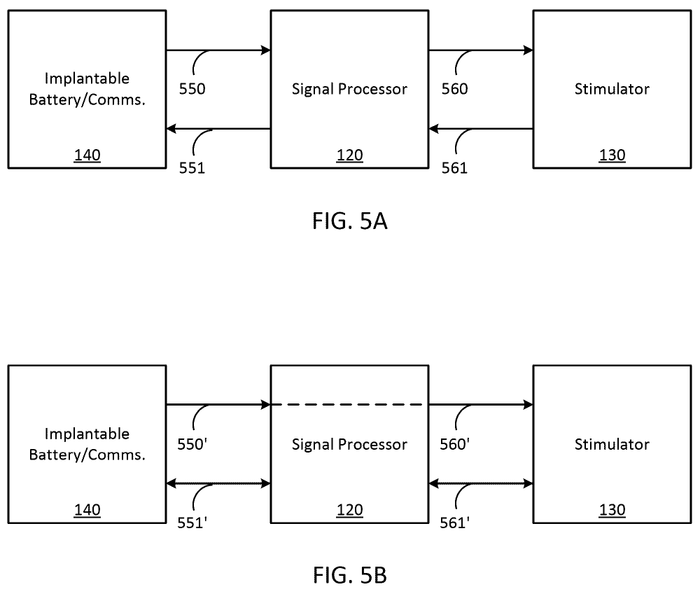

FIGS. 5A and 5B show high-level schematic diagrams illustrating example communication configurations between an implantable battery and/or communication module, a signal processor, and a stimulator. FIG. 5A shows a high-level schematic diagram illustrating an exemplary communication configuration between an implantable battery and/or communication module, a signal processor, and a stimulator. In the example of FIG. 5A, the implantable battery and/or communication module 140 is in two-way communication with the signal processor 120. For instance, the implantable battery and/or communication module 140 can communicate power and/or data signals 550 to the signal processor 120. In some examples, the power and data signals 550 can be included in a single signal generated in the implantable battery and/or communication module 140 and transmitted to the signal processor 120. Such signals can include, for example, a digital signal transmitted with a particular clock rate, which in some embodiments, can be adjustable, for example, via the implantable battery and/or communication module 140.

In some embodiments, the signal processor 120 can communicate information to the implantable battery and/or communication module 140 (e.g., 551), for example, feedback information and/or requests for more power, etc. The implantable battery and/or communication module 140 can, in response, adjust its output to the signal processor 120 (e.g., an amplitude, duty cycle, clock rate, etc.) in order to accommodate for the received feedback (e.g., to provide more power, etc.). Thus, in some such examples, the implantable battery and/or communication module 140 can communicate power and data (e.g., 550) to the signal processor 120, and the signal processor 120 can communicate various data back to the implantable battery and/or communication module 140 (e.g., 551).

In some embodiments, similar communication can be implemented between the signal processor 120 and the stimulator 130, wherein the signal processor 120 provides power and data to the stimulator 130 (e.g., 560) and receives data in return from the stimulator 130 (e.g., 561). For example, the signal processor 120 can be configured to output signals (e.g., power and/or data) to the stimulator 130 (e.g., based on received inputs from a middle ear sensor or other device) via a similar communication protocol as implemented between the implantable battery and/or communication module 140 and the signal processor 120. Similarly, in some embodiments, the stimulator can be configured to provide feedback signals to the signal processor, for example, representative of an executed stimulation process. Additionally or alternatively, the stimulator may provide diagnostic information, such as electrode impedance and neural response telemetry or other biomarker signals.

While signals 550, 551, 560, 561 are represented in FIG. 5A via a single line, in various embodiments, such signals can be communicated using one or more conductors. In some examples, such signals are communicated via a plurality of conductors, and in some such examples, communicating a signal comprises communicating a signal and its inverse so that no net charge travels between components due to the communication. Capacitive coupling on connecting leads can also be used to block DC charge from flowing during communication. Example configurations for such communication is described in U.S. Patent Publication No. US2020/0269035A1, entitled IMPLANTABLE COCHLEAR SYSTEM WITH INTEGRATED COMPONENTS AND LEAD CHARACTERIZATION, which is incorporated herein by reference.

FIG. 5B is an alternative high-level schematic diagram illustrating an exemplary communication configuration between an implantable battery and/or communication module, a signal processor, and a stimulator. In the example of FIG. 5B, the implantable battery and/or communication module 140 provides signals (e.g., 550′) to the signal processor 120 via a first communication link and is further in two-way communication for providing additional signals (e.g., 551′) with the signal processor 120. In the example of FIG. 5B, the implantable battery and/or communication module 140 can provide power signals (e.g., 550′) to the signal processor 120 via a communication link and otherwise be in two-way data communication (551′) with the signal processor 120 via a second communication link. In some such examples, the power (550′) and data (551′) signals can each include digital signals. However, in some embodiments, the power and data signals are transmitted at different clock rates. In some examples, the clock rate of the data signals is at least one order of magnitude greater than the clock rate of the power signals. For example, in an exemplary embodiment, the power signal is communicated at a clock rate of approximately 30 kHz, while the data communication occurs at a clock rate of approximately 1 MHz. Similarly to the embodiment described in FIG. 5A, in some examples, the clock rate can be adjustable, for example, via the implantable battery and/or communication module 140.

As described with respect to FIG. 5A, in some embodiments, the signal processor 120 can communicate information to the implantable battery and/or communication module 140, for example, feedback information and/or requests for more power, etc. (e.g., data signals 551′). The implantable battery and/or communication module 140 can, in response, adjust the power and/or data output to the signal processor 120 (e.g., an amplitude, duty cycle, clock rate, etc.) in order to accommodate for the received feedback (e.g., to provide more power, etc.).

In some embodiments, similar communication can be implemented between the signal processor 120 and the stimulator 130, wherein the signal processor 120 provides power and data to the stimulator 130 and receives data in return from the stimulator 130. For example, the signal processor 120 can be configured to output power signals (e.g., 560′) and data signals (e.g., 561′) to the stimulator 130 (e.g., based on received inputs from a middle ear sensor or other device). Such communication can be implemented via a similar communication protocol as implemented between the implantable battery and/or communication module 140 and the signal processor 120. In some examples, the power signals provided to the stimulator 130 (e.g., 560′) are the same signals (e.g., 550′) received by the signal processor 120 from the implantable battery and/or communication module 140. Additionally, in some embodiments, the stimulator 130 can be configured to provide feedback signals to the signal processor 120 (e.g., 561′), for example, representative of an executed stimulation process.

Similar to FIG. 5A discussed above, example configurations for such communication is described in U.S. Patent Publication No. US2020/0269035, entitled IMPLANTABLE COCHLEAR SYSTEM WITH INTEGRATED COMPONENTS AND LEAD CHARACTERIZATION, which is incorporated herein by reference.

Various systems and methods can be employed provide communication between system components. In some examples, data can be communicated to the implantable battery and/or communication module from an external component, such as a programmer as shown in FIG. 1. In an exemplary process, a programmer, such as a clinician's computer, can be used to communicate with a patient's fully implanted system via the implantable battery and/or communication module, which can communicate information to other system components, such as via lead 190.

During such processes, a clinician can communicate with the signal processor, and, in some cases, with other components via the signal processor. For example, the clinician can cause the signal processor to actuate an electrical and/or an acoustic stimulator in various ways, such as using various electrical stimulation parameters, combinations of active contact electrodes, various acoustic stimulation parameters, and various combinations thereof. Varying the stimulation parameters in real time can allow the clinician and patient to determine effectiveness of different stimulation techniques for the individual patient. Similarly, the clinician can communicate with the signal processor to update transfer function. For example, the clinician can repeatedly update the transfer function signal processor while testing the efficacy of each one on the individual patient. In some examples, combinations of stimulation parameters and signal processor transfer functions can be tested for customized system behavior for the individual wearer.

In some embodiments, various internal properties of the system may be tested. For instance, various impedance values, such as a sensor, stimulator, or electrode impedance can be tested such as described in U.S. Patent Publication No. 2015/0256945, entitled TRANSDUCER IMPEDANCE MEASUREMENT FOR HEARING AID and U.S. Patent Publication No. 2022/0313997, entitled COCHLEAR IMPLANT SYSTEM WITH ELECTRODE IMPEDANCE DIAGNOSTICS, each of which is assigned to the assignee of the instant application, the relevant portions of which are incorporated by reference herein.

As described elsewhere herein, in various embodiments, the processor generally receives an input signal, processes the signal, and generates a stimulation signal, which can be applied via a stimulator. The stimulator can be in communication with the signal processor via one or more leads (e.g., as shown in FIGS. 1 and 4) or can be integrated into a single housing with the signal processor, for example, as described in U.S. Patent Publication No. US2020/0269035, which is incorporated herein by reference. In some such embodiments, the input signal received via the signal processor is generated by an implantable sensor, such as a middle ear sensor.

However, such sensors often measure or otherwise receive some stimulus that is converted into an output that is read and processed by the signal processor. For example, some middle ear sensors may produce a different output signal for a given stimulus depending on a variety of factors, such as variability in a wearer's inner-ear anatomy and motion. Thus, the output of a sensor for a given input may be not predictable while designing a system, especially across a range of frequencies.

Various signal processing examples for adapting to variability in a sensor frequency response are described in U.S. Patent Publication No. US2020/0269035, which is incorporated herein by reference. As described elsewhere herein, while many examples show a middle ear sensor being in communication with an implanted signal processor, in various embodiments, one or more additional or alternative input sources can be included. For instance, in some embodiments, a microphone can be implanted under a user's skin and can be placed in communication with the signal processor (e.g., via a detachable connector such as 171). The signal processor can receive input signals from the implanted microphone and provide signals to the stimulator based on the received input signal and the signal processor transfer function.

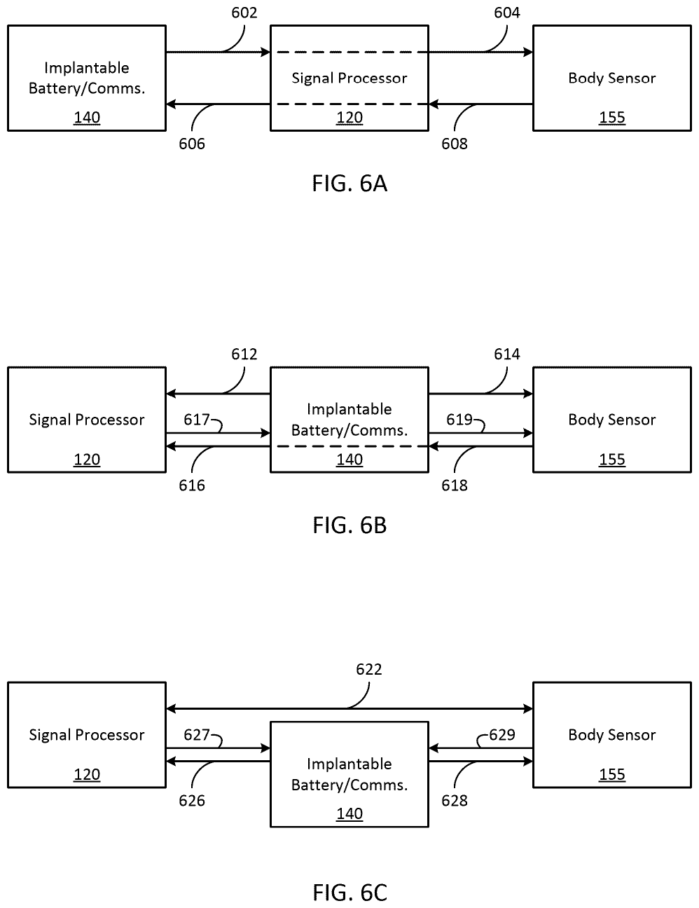

As described elsewhere herein, systems can include one or more body sensors configured to output a body parameter signal representative of one or more body parameters sensed by the body sensor. FIGS. 6A-6C show example communication arrangements between an example body sensor, signal processor, and implantable battery and/or communication module.

FIG. 6A shows a high-level schematic showing a communication configuration between an example body sensor, signal processor, and implantable battery and/or communication module. As shown, an implantable battery and/or communication module 140 is in communication with signal processor 120. As described elsewhere herein, in some embodiments, implantable battery and/or communication module 140 can provide power and data to signal processor 120, shown here, via signal 602. In the illustrated example, signal processor 120 provides signal 604 to body sensor 155. Signal 604 can include a data signal, for example, to program one or more aspects of body sensor 155 or request data therefrom. Additionally or alternatively, in some examples, signal 604 provides electrical power to body sensor 155. In some such examples, the electrical power provided to body sensor 155 via signal 604 is the same electrical power provided from the implantable battery and/or communication module 140 to signal processor 120 via signal 602.

In the example of FIG. 6A, body sensor 155 provide signal 608 to the signal processor 120. In some examples, signal 608 includes a body parameter signal representative of one or more body parameters sensed or otherwise determined by body sensor 155, such as a temperature sensor, pressure sensor, electrode, or other appropriate sensors. In various examples, signal 608 can provide a continuous body parameter signal indicative of the body parameter. In other examples, signal 608 can provide a body parameter signal to the signal processor 120 according to a preprogrammed schedule or upon request from the signal processor 120.

In the example of FIG. 6A, the signal processor 120 communicates signal 606 to implantable battery and/or communication module 140. In some examples, the signal processor 120 communicates the body parameter signal to the implantable battery and/or communication module 140, for example, for the implantable battery and/or communication module 140 to output via a wireless communication interface. In some examples, the signal processor 120 relays the body parameter signal received from the body sensor 155 to the implantable battery and/or communication module 140 (e.g., as shown via signals 608 and 606 and broken line). In other examples, the signal processor 120 outputs a signal representative of the body parameter to the implantable battery and/or communication module 410 via signal 606 without necessarily precisely duplicating signal 608 received from body sensor 155.

In some examples, in a configuration such as shown in FIG. 6A, a request by the signal processor via signal 604 for a body parameter signal from the body sensor 155 is initiated by a command signal via implantable battery and/or communication module 140. For example, in an example embodiment, an external device (e.g., a smartphone, computer, system programmer, system charger, etc.) can be in wireless communication with the implantable battery and/or communication module 140 via wireless communication interface (e.g., Bluetooth). The external device can send a signal to the implantable battery and/or communication module 140 requesting body parameter information. The implantable battery and/or communication module 140 can instruct the signal processor 120 via signal 602 to request a body parameter signal from the body sensor 155, and signal processor sends such a request via signal 604. In some such examples, the body sensor 155 can output a body parameter signal representative of a body parameter to signal processor 120 via signal 608. Signal processor 120 can output a signal representative of the body parameter to the implantable battery and/or communication module 140 via signal 606, and the implantable battery and/or communication module 140 can output a representation of the body parameter via a wireless communication interface.

In the embodiment of FIG. 6A, implantable battery and/or communication module 140 and signal processor 120 can be connected via a first lead (e.g., lead 190 in FIG. 1), and body sensor 155 and signal processor 120 can be connected via a second lead (e.g., lead 197 in FIG. 1). However, in some such embodiments, no lead connects implantable battery and/or communication module 140 and body sensor 155.

FIG. 6B shows an alternate communication arrangement of the signal processor, implantable battery and/or communication module, and body sensor. In the illustrated example of FIG. 6B, implantable battery and/or communication module 140 provides signal 612 to signal processor 120 and signal 614 to body sensor 155. In some examples, implantable battery and/or communication module provides electrical power to both signal processor 120 and body sensor 155 via signals 612 and 614, respectively. In some embodiments, equal power signals are sent to signal processor 120 and body sensor 155, and in some such examples, signals 612 and 614 comprise parallel power signals.

In the example of FIG. 6B, the implantable battery and/or communication module 140 is in bidirectional communication with signal processor 120 and body sensor 155. As shown, the implantable battery and/or communication module 140 can receive data from signal processor via signal 617 and provide data to signal processor via signal 616. Additionally, implantable battery and/or communication module 140 can communicate information to body sensor 155 via signal 619 and receive information from body sensor 155 via signal 618.

In some embodiments, implantable battery and/or communication module can request information related to a body parameter from body sensor 155 by sending a request via signal 619, and can receive a body parameter signal from body sensor 155 via signal 618. As described elsewhere herein, in some examples, implantable battery and/or communication module 140 can output information representative of the body parameter via a wireless communication interface. Additionally or alternatively, implantable battery and/or communication module 140 can communicate a signal representative of the body parameter to the signal processor 120 via signal 616. In some embodiments, the implantable battery and/or communication module 140 is configured to directly relay the body parameter signal from the body sensor 155 to the signal processor 120, such as shown by the broken line in FIG. 6B. In other examples, the implantable battery and/or communication module 140 is configured to output a signal representative of the body parameter, but not necessarily precisely duplicating the body parameter signal received at 618.

In the embodiment of FIG. 6B, implantable battery and/or communication module 140 and signal processor 120 can be connected via a first lead (e.g., lead 190 in FIG. 1), and body sensor 155 and implantable battery and/or communication module 140 can be connected via a second lead (e.g., lead 196 in FIG. 1). However, in some such embodiments, no lead connects signal processor 120 and body sensor 155.

Similar to as described elsewhere herein, in some examples, some signals can be combined. For instance, in some embodiments, implantable battery and/or communication module 140 can provide data and power together via a single signal. In an example embodiment, implantable battery and/or communication module 140 can communicate power and data to signal processor 120 via signal 612. Other signals can similarly be configured to include power and data.

FIG. 6C shows another alternate communication arrangement of the signal processor, implantable battery and/or communication module, and body sensor. In the illustrated example of FIG. 6C, implantable battery and/or communication module 140 provides signal 626 to signal processor 120 and signal 628 to body sensor 155. In some examples, implantable battery and/or communication module 140 provides electrical power to both signal processor 120 and body sensor 155 via signals 626 and 628, respectively. In some embodiments, equal power signals are sent to signal processor 120 and body sensor 155, and in some such examples, signals 626 and 628 comprise parallel power signals.

As shown in FIG. 6C, signal processor is in communication with body sensor 155 via signal 622. In some embodiments, signal 622 provides body parameter information to signal processor 120 from the body sensor 155. In some such embodiments, the body sensor 155 is configured to continually output a body parameter signal to the signal processor 120.

In some examples, signal processor 120 and body sensor 155 are in bidirectional communication via signal 622. In some such examples, signal processor 120 is configured to send a signal to body sensor 155 in order to request body parameter information from the body sensor 155.

In the illustrated example, implantable battery and/or communication module 140 is configured to receive information from the body sensor 155 via signal 629. In some examples, body sensor 155 is configured to continually output a body parameter signal to the implantable battery and/or communication module 140, and in other examples, the body sensor 155 is configured to send a body parameter signal to the implantable battery and/or communication module 140 upon request. In some embodiments, implantable battery and/or communication module 140 is configured to output a wireless signal representative of a body parameter measured by body sensor 155. In some embodiments, implantable battery and/or communication module 140 can receive a command from an external device in wireless communication therewith to output a wireless signal representative of the body parameter and, in some examples, to request a body parameter signal from the body sensor 155.

In the example of FIG. 6C, signal processor 120 is in communication with implantable battery and/or communication module 140 via signal 627. As described elsewhere herein, in some embodiments, implantable battery and/or communication module 140 can be configured to provide power and data to signal processor 120 via the same signal (e.g., via signal 626). Additionally or alternatively, implantable battery and/or communication module 140 can be configured to provide data to the signal processor 120 via a signal separate from the power provided to the signal processor 120.

In the embodiment of FIG. 6C, implantable battery and/or communication module 140 and signal processor 120 can be connected via a first lead (e.g., lead 190 in FIG. 1), body sensor 155 and implantable battery and/or communication module 140 can be connected via a second lead (e.g., lead 196 in FIG. 1), and signal processor 120 and body sensor 155 can be in communication via a third lead (e.g., lead 197 in FIG. 1).

It will be appreciated that, while each signal in each of FIGS. 6A, 6B, and 6C is shown as a single signal, such signals can be transmitted via one or more conductors, for example, including transmitting a signal and its inverse, such as described in U.S. Patent Publication No. US2020/0269035, which is incorporated herein by reference. Additionally or alternatively, signals can serve multiple functions, such as transmitting both power and data signals, such as described in U.S. Patent Publication No. US2020/0269035, which is incorporated herein by reference. Additionally or alternatively, in some examples, bidirectional communication (e.g., signals 602 and 606 between signal processor 120 and implantable battery and/or communication module 140 in FIG. 6A) can be provided via a single lead (e.g., lead 190 connecting signal processor 120 and implantable battery and/or communication module 140 in FIG. 1) having one or more conductors.

In some examples, leads connecting components to provide communication therebetween can include any number of conductors in order to provide the desired communication. For example, a lead can include a plurality of conductors providing bidirectional communication and or to provide a signal and its inverse via the length of the lead in order to provide net charge neutral communication.

Various communication configurations are shown in FIGS. 6A-C. Such configurations are shown as illustrative examples and do not limit the way that various such components can be arranged to communicate. In various examples, signals shown in the illustrated examples can be implemented as bidirectional communication signals, can be combined with other signals, such as power signals, or other arrangements. In some examples, one or more of the illustrated signals can be communicated via a wireless communication link, such as via Bluetooth communication. For example, in some embodiments, body sensor 155 can be configured to wireless communicate information to the signal processor 120 and/or the implantable battery and/or communication module 140.

Additionally or alternatively, in some examples, various illustrated signals can be excluded. For instance, in some embodiments, a body sensor 155 does not require any electrical power to operate and provide a body parameter signal. In some such examples, implantable battery and/or communication module 140 does not provide electrical power to the body sensor 155 (e.g., directly or via signal processor 120). In some such examples, body sensor 155 operates without power. In other examples, body sensor 155 can include a built-in power source, such as one or more batteries.

Various non-limiting communication configurations are shown in FIGS. 6A-C. Various implementations are possible and within the scope of this disclosure. In various embodiments, one or more body sensors 155 are in communication with an implantable battery and/or communication module 140, either directly (e.g., via lead 196 in FIG. 1) or via the signal processor 120 (e.g., via leads 197 and 190 in FIG. 1). In some examples, one or more body sensors 155 are in communication with a signal processor 120, either directly (e.g., via lead 197 in FIG. 1) or via implantable battery and/or communication module 140 (e.g., via leads 169 and 190 in FIG. 1). In some examples, one or more body sensors 155 are in direct communication with signal processor 120 (e.g., via lead 197) and in direct communication with implantable battery and/or communication module 140 (e.g., via lead 196).

In some examples, one or more body sensors 155 are configured to receive electrical power from implantable battery and/or communication module 140 or from signal processor 120. In other examples, one or more body sensors 155 do not require power applied thereto in order to generate and output a body parameter signal.

In some embodiments, different body sensors 155 can be arranged in different configurations from one another, for example, with one or more body sensors in direct communication with implantable battery and/or communication module 140 and one or more additional body sensors in direct communication with signal processor 120. In general, any number of body sensors can be used, and each body sensor can be in communication with signal processor 120 and/or implantable battery and/or communication module 140 in various ways such as described herein.

In some examples, the signal processor 120 can receive body parameter information directly from one or more body sensors (e.g., receiving a body parameter signal directly therefrom) or indirectly, such as from the implantable battery and/or communication module 140. Additionally or alternatively, in some examples, implantable battery and/or communication module 140 can receive body parameter information directly from one or more body sensors (e.g., receiving a body parameter signal directly therefrom) or indirectly, such as from the signal processor 120.

As described, in some embodiments, signal processor 120 is configured to receive body parameter information representative of a body parameter sensed by a body sensor. In some such examples, the signal processor is configured to generate a stimulation signal based on the body parameter information. For example, in some embodiments, the signal processor is configured to generate a stimulation signal that mimics an audio signal that “reads” the body parameter for the wearer to perceive. For example, the signal processor could generate a stimulation signal for output to the stimulator to cause the stimulator to output an electrical stimulus causing the wearer to perceive an artificial “sound” that reads the body parameter to the wearer. Such stimulation could be triggered, for example, in response to a command by the wearer or a clinician, such as via an external device in communication with the implanted system (e.g., via wireless communication with the implantable battery and/or communication module).

Additionally or alternatively, in some examples, implantable battery and/or communication module 140 is configured to receive body parameter information representative of a body parameter sensed by a body sensor. In some such examples, the implantable battery and/or communication module 140 is configured to output body parameter information to an external device, such as via a wireless communication interface. Such communication could be triggered, for example, in response to a command by the wearer or a clinician, such as via the external device that receives the communication or another external device in communication with the implanted system.

In an example embodiment, an external device (e.g., a smartphone, charger, programmer, etc.) receives an input via a user interface requesting body parameter information from a body sensor. In some examples, the input identifies a means by which the body parameter is communicated, such as being communicated wirelessly to an external device or being “read” to a wearer via a generated stimulation signal representative of the body parameter information. In response to the received input, the external device can communicate wirelessly with an implanted cochlear implanted system (e.g., via a wireless communication interface in the implantable battery and/or communication module) to request the data be communicated (e.g., wirelessly or via stimulation).

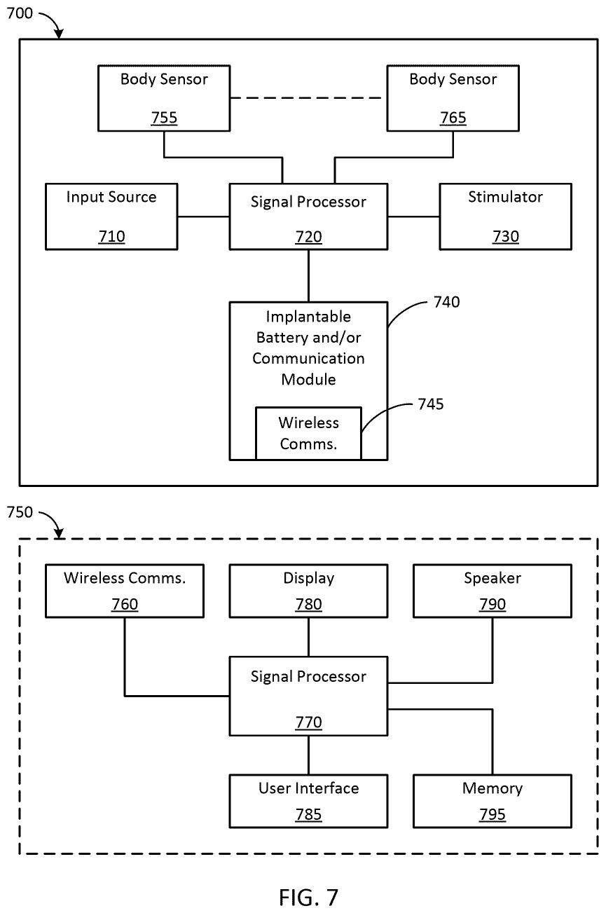

FIG. 7 shows a schematic diagram of an example cochlear implant and analysis system including an exemplary implantable cochlear implant system 700 and an external device 750. In some examples, cochlear implant system 700 is fully implantable. Cochlear implant system 700 can be in wireless communication with an external device 750 and can transmit signals to the external device 750. In an example operation, the external device 750 can receive and store signals from the cochlear implant system 700 while the cochlear implant system is operating. Further, the external device 750 can receive and store signals from an input source external to the cochlear implant system 700 while the cochlear implant system is operating. The external device 750 can optionally compare signals from the cochlear implant system with signals from the input source. In some examples, adjustments to the cochlear implant system 700 can be made based on the comparison of such signals.

The cochlear implant system 700 includes an input source 710, such as a microphone, middle ear sensor, inner ear sensor, or the like, in communication with signal processor 720. The input source 710 can be a sensor configured to receive an input representative of ambient sound and generate an input signal representative of the received input. For example, as described elsewhere herein, the input source 710 can be an inner ear sensor, middle ear sensor, microphone, or other sensor which can receive sound and generate a representative input signal. In operation, once generated, the input signal representative of the sound can be sent to the signal processor 720.

The signal processor 720 can be representative of any element or combination of elements which can receive an input signal from the input source 710 and generate an output signal based on the input signal. In some examples, the stimulation signal can be based on the input signal received by signal processor 720 and on a transfer function. In some examples, the signal processor comprises one or more additional processing components other than the signal processor which can perform further processing on the received input signals. In some examples, the processing performed by the signal processor 720 includes one or more intermediate steps used to generate the stimulation signal based on a signal received from input source 710. For instance, in some examples, one or more such intermediate steps include analog processing, digital processing, and/or generating a stimulation signal via a transfer function as described in U.S. Patent Publication No. US2020/0269035, which is incorporated herein by reference.

In addition to being in communication with the input source 710, the signal processor 720 is in communication with a stimulator 730. As described elsewhere herein, in some embodiments, signal processor 720 can generate a stimulation signal and output the stimulation signal to stimulator 730. The stimulator 730 can be configured to apply electrical signals to a patient's cochlear tissue via one or more electrodes based on the stimulation signal.

Further, signal processor 720 is in communication with an implantable battery and/or communication module 740. As described elsewhere herein, power and/or data can be sent between the signal processor 720 and the implantable battery and/or communication module 740. In some examples, the implantable battery and/or communication module 740 sends power to the signal processor 720 and the signal processor 720 sends data to the implantable battery and/or communication module 740. In some examples, the implantable battery and/or communication module 740 sends both power and data to the signal processor 720.

The cochlear implant system 700 of FIG. 7 further includes body sensors 755 and 765. While the system as shown includes two body sensors, systems can include one, two, three, or more body sensors in communication with one or more other system components. In the illustrated example, body sensor 755 is in communication with signal processor 720. As described elsewhere herein, in some embodiments, body sensor 755 is configured to output a body parameter signal representative of one or more body parameters to signal processor 720.

In some embodiments, the signal processor is configured to process the body parameter signal and generate an output configured to present body parameter information representative of a body parameter. For instance, in an example embodiment, signal processor 720 is configured to generate a stimulation signal representative of the body parameter and output such stimulation signal to the stimulator such that a wearer can perceive information regarding the body parameter via the stimulation. Additionally or alternatively, the signal processor 720 can output body parameter information to the implantable battery and/or communication module 740 such as described elsewhere herein.

While shown as being in communication with the signal processor 720, as described elsewhere herein, in various embodiments, body sensor 755 can additionally or alternatively be in communication with the implantable battery and/or communication module 740.

The example cochlear implant system of FIG. 7 includes a second body sensor 765. Second body sensor 765 can be configured to output a second body parameter signal. The second body parameter signal can be representative of the same body parameter as the body parameter signal output from body sensor 755, for example, as measured at a different location in the body. In other examples, second body parameter signal can represent one or more additional body parameters. Similar to described above with respect to body sensor 755, body sensor 765 can provide a body parameter signal to the signal processor 720, which can output one or more signals representative thereof (e.g., via a stimulation signal to stimulator 730, as a signal to implantable battery and/or communication module 740, etc.). Additionally or alternatively, body sensor 765 can output a body parameter signal to implantable battery and/or communication module 740. As described herein, in some examples, a first body sensor can output a body parameter signal to signal processor 720 while a second body sensor can output a body parameter signal to implantable battery and/or communication module 740.

In the example of FIG. 7, cochlear implant system 700 can be configured to output one or more signals to an external device 750. In some embodiments, to output signals from the cochlear implant system 700 to the external device, the implantable battery and/or communication module 740 of the cochlear implant system 700 can include a wireless communication interface 745. The wireless communication interface 745 can communicate with the external device 750 which can include a wireless communication interface 760 capable of wireless communication with wireless communication interface 745. The wireless communication interface 745 of the cochlear implant system 700 can communicate with a wireless communication interface 760 of the external device 750 to exchange data.

While described as sending or outputting data to the external device 750, in some examples, the wireless communication interface 745 can receive signals from the external device 750. In some examples, implantable battery and/or communication module 740 can send received signals to the signal processor 720. The wireless communication between the two wireless communication interfaces 745, 760 can comprise a variety of communication protocols, such as Wi-Fi, Bluetooth, NFC and/or other data transmission protocols.

While the example of FIG. 7 illustrates the wireless communication module 745 as part of the implantable battery and/or communication module 740, the wireless communication module 745 can be separate. For instance, in some examples, the wireless communication module can be in communication with the implantable battery and/or communication module but be located in a separate housing. In some examples, the signal processor 720 can include a wireless communication module and output signals to the external device 750 without going through the implantable battery and/or communication module.

As described elsewhere herein, in some examples, body parameter information can be output wirelessly from an implanted system (e.g., from wireless communication interface 745) to an external device (e.g., 750). In some examples the wireless communication interface 745 of the cochlear implant system 700 can output an altered signal representative of, for example, a body parameter signal. For instance, in various examples, a body parameter signal can be downsampled and/or transformed (e.g., via a Fast Fourier Transform) to create an altered signal still representative of the body parameter.

In the illustrated example of FIG. 7, external device 750 includes a wireless communication interface 760, an external signal processor 770, a display 780, a user interface 785, a speaker 790, and memory 795. In some embodiments, the external device 750 is configured to wirelessly receive information from the wireless communication interface 745 of the implantable battery and/or communication module 740, such as body parameter information representative of a body parameter sensed by a body sensor (e.g., 755). In some embodiments, external device can display body parameter information representative of the body parameter via display 780. Additionally or alternatively, external device 750 can output an audible signal including body parameter information via speaker 790. In some examples, such outputting of the body parameter information is controlled by external signal processor 770 in communication with the wireless communication interface 760 and can receive body parameter information therefrom.

In addition or alternatively to outputting a representation of body parameter information (e.g., visually via display 780 and/or audibly via speaker 790), external device 750 can be configured to store body parameter information is memory 795. In some examples, the external device 750 can be configured to access and store information, such as body parameter information, in a separate memory over a network, such as in cloud-based memory. External device 750 can be configured to access and display previously-stored body parameter information from memory, for example, to compare newly-acquired body parameter information to historic information. In some embodiments, body parameter information can be combined or analyzed in combination to make determinations regarding the wearer.

The external device 750 of FIG. 7 includes a user interface 785 in communication with the external signal processor 770. In some examples, user interface 785 is configured to receive an input from a user to control one or more aspects of the operation of the external device 750 and/or cochlear implant system 700. For example, in some embodiments, external device 750 can receive a command via the user interface 785 to measure a body parameter of a wearer of the cochlear implant system 700. The external signal processor 770 can be configured to receive such input from the user interface 785 and send a command to the cochlear implant system 700 via wireless communication interface 745.

The cochlear implant system 700 can receive the command via wireless communication interface 745 and access body parameter information from one or more body sensors such as described herein. Cochlear implant system can be configured to output body parameter information as described herein, such as by generating a stimulation signal representative of the body parameter information and/or by outputting body parameter information via wireless communication interface 745. In some examples, the mechanism by which the cochlear implant system outputs body parameter information is controlled by external device 750. For example, a user can, via user interface 785 instruct the cochlear implant system to output body parameter information in a certain way.

While the illustrated embodiment includes the listed features, various external devices can include more or fewer components than that shown in FIG. 7. In various embodiments, external device 750 can be any device or series of devices that can receive information from the cochlear implant system 700. For example, the external device can be a computer, smartphone, tablet, or the like. In some cases, various components shown as part of external device 750 can be distributed among multiple external devices operating in concert.

As described elsewhere herein, the wireless communication interface 760 of external device 750 can communicate with the wireless communication interface 745 of implantable battery and/or communication module 740 to send and/or receive signals. In some embodiments, the wireless communication interface 760 of the external device 750 can be integrated in with the external signal processor 770 or alternatively, can be separate from and communicate with the external signal processor 770.

In some embodiments, the external signal processor 770 of the external device 750 is in communication with the wireless communication interface 760 and can receive signals from the cochlear implant system 700 and/or send signals to the cochlear implant system 700 via the wireless communication interface 760. The external signal processor 770 can comprise one or more signal processors (e.g., digital signal processors), one or more microprocessors, microcontrollers, application specific integrated circuits (ASICs), or the like. In FIG. 7, the signal processor of the cochlear implant system can be considered a first signal processor 720 with the signal processor of the external device being considered an external signal processor 770.

As described, in some embodiments, memory 775 of the external device 750 can receive and store information from the external signal processor 770 such as body parameter information from the cochlear implant system 700. The memory 775 can comprise any type of computer memory including volatile memory, non-volatile memory, random access memory (RAM), read only memory (ROM), and the like. In some examples, the memory 775 comprises a non-transitory computer readable medium and can comprise instructions that can be executed by the external signal processor 770. In some examples, the memory is integrated with the external signal processor 770 while in some examples, the memory is separate from, but in communication with, the external signal processor 770.

In some embodiments, the external device 750 includes display 780 in communication with external signal processor 770. In some examples, the display 780 is separate from the external device 750 but is in communication with the external device 750 (e.g., via wireless communication interface 760). The display 780 can comprise any type of display (e.g., LCD, LED, OLED) and can include computer monitors, televisions, and handheld device displays.

As described elsewhere, in some examples, the external device 750 can include a user interface to enable user input. For example, the display 780 of the external device can be a touch-sensitive display with a graphical user interface. A touch-sensitive display can allow a user to interact with the graphical user interface of the external device 750 and provide inputs to the external device 750. In some examples, the external device can receive user inputs via a mouse, keyboard, and/or other human interface device. Inputs provided by a user to the external device 750 can control many different aspects of the external device including the wireless communication interface 760, external signal processor 770, memory 775, display 780, and/or speaker 790.

The external device 750 of FIG. 7 also includes a speaker 790 in communication with external signal processor 770. In some examples, speaker 790 is separate from external device 750, but is in communication with the external device 750. Speaker 790 can be any type of speaker which produces audio output and can produce audio outputs representative of any signals which it receives. The speaker 790 can receive signals from the external signal processor 770 and generate an audio representation of the received signals. In some examples, the external signal processor 770 outputs a signal to the speaker to cause the speaker to output an audio representation of a given signal. For example, the speaker 790 can receive a signal from the external signal processor 770 and produce a pure tone, a phoneme, a word, a phrase, or other sound corresponding to the received signal.

While the wireless communication interface 760, the memory 775, the display 780, and the speaker 790 are all illustrated as part of the external device 750, in some examples, one or more of the listed components are separate from the external device. In some such examples, the separate components are still in communication with the external signal processor 770 (e.g., via wired or wireless communication).

In some embodiments, body parameter information can be compared to one or more threshold values or ranges stored in a memory, such as memory 775 in external device and/or an implanted memory, such as a memory associated with the signal processor 720 or implantable battery and/or communication module 740. In some such examples, if the body parameter information is outside of a predetermined range (e.g., below a lower threshold and/or above an upper threshold), the system can be configured to output an alert to a user that the body parameter is outside of a predetermined range.

In some examples, implanted signal processor is configured to periodically monitor body parameter information from one or more body sensors (e.g., 755) and compare the body parameter information to one or more thresholds. For instance, signal processor 720 can receive body parameter information from body sensor 755 and compare the body parameter information to one or more thresholds at a rate of once per minute, once per hour, once per day, once per week, once per month, or at some other rate. In some examples, the system does not necessarily output a perceivable signal at each measurement instance. In various examples, body parameter information can be stored in a memory for future access and/or can be available for real-time analysis upon request. In some cases, a system can be configured to perform multiple body parameter measurements using multiple body sensors, and in some such examples, different body parameters can be measured at different rates.

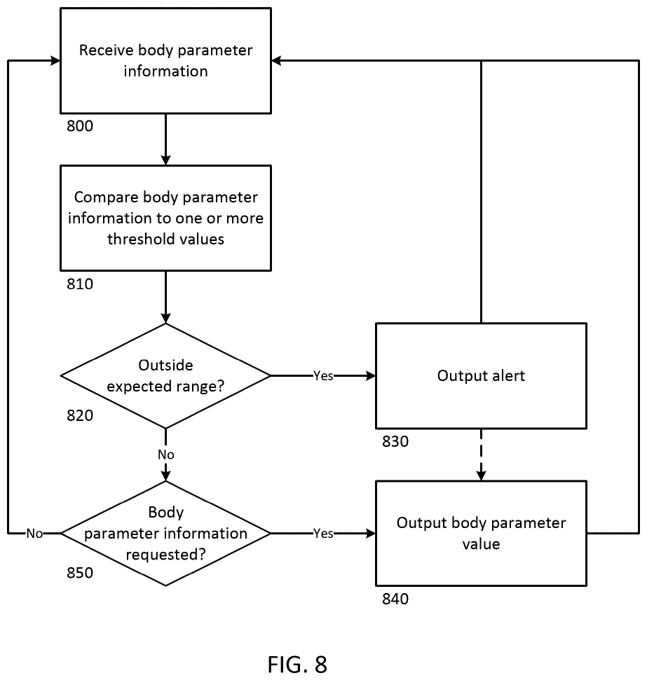

FIG. 8 is a process flow diagram showing an example process for monitoring a body parameter. In the illustrated example, the process comprises receiving body parameter information (800) and comparing the body parameter information to one or more threshold values (810). In some examples, comparing the body parameter information to one or more threshold values comprises comparing a body parameter to a single threshold, such as a minimum threshold value or a maximum threshold value. In other examples, comparing the body parameter information to one or more threshold values comprises determining whether a body parameter is within a predetermined range of values, such as between a minimum threshold value and a maximum threshold value.

The process of FIG. 8 includes determining whether the body parameter information is outside of an expected range (820). In various examples, the determining whether the body parameter information is outside of an expected range can include determining if the body parameter is above a threshold value, below a threshold value, or between two threshold values.

In the example of FIG. 8, if the body parameter is outside of the expected range, the process includes outputting an alert (830). In some examples, outputting an alert comprises generating a stimulation signal and providing electrical stimulation to the wearer to alert the wearer of the body parameter. Additionally or alternatively, in some examples, outputting an alert comprises causing an external device to output an alert, such as via a display (e.g., 780) and/or speaker (e.g., 790).

In some embodiments, the process further includes outputting the body parameter value (840). In various embodiments, outputting an alert can include outputting information regarding the body parameter, such as the body parameter value, the range outside which the body parameter value lies that triggered the alert, recommended actions, or other information.

In the example of FIG. 8, if the body parameter is not outside the expected range, the process can include determining whether body parameter information has been otherwise requested (850). For example, as described elsewhere herein, in some embodiments, a request for body parameter information can be made via an external device. If body parameter information has been requested, the process includes outputting a body parameter value (840), such as by a stimulation signal for via an external device as described herein.

As shown, in some examples, if a body parameter is not outside of an expected range in step 820 and no body parameter information has been requested in step 850, nothing is output and the process repeats. Similarly, in some embodiments, even if the body parameter is outside of an expected range and an alert is output (830), the process repeats. In some embodiments, the process repeats according to a preset schedule or analysis frequency, such as once per minute, once per hour, once per day, once per week, etc.

Thus, in some embodiments, a system can be configured to silently analyze body parameter information and compare the body parameter information to one or more threshold values to determine if the body parameter is outside of an expected range. If not, the system continues to silently monitor the body parameter. If at some point the body parameter falls outside of the expected range, one or more alerts can be presented. Meanwhile, the system can be configured to output body parameter information to a user upon request whether or not a body parameter falls outside of an expected range.

As described herein, various body sensors are possible. In various examples, body sensors can be configured to output body parameter information representative of heart rate, body temperature, an electrocardiogram, an electroencephalogram (EEG), blood oxygen levels, or others, such as described elsewhere herein.

In some embodiments, a body sensor can be configured to detect acoustic stimuli. For example, a body sensor can include one or more implanted microphones or other devices configured to output a signal representative of an acoustic stimulus. In some such embodiments, body parameter information can include acoustic information representative of one or more acoustic stimuli generated by the body. For instance, in an example implementation, body parameter information can include information representative of sounds generated by the body, such as snoring or respiratory sounds. A signal processor can be configured to receive information from, for example, an implanted microphone. In some examples, the signal processor is configured to process the received sounds to detect one or more detectable acoustic signatures, for example, to identify snoring detected at the body parameter sensor.

As described elsewhere herein, in some embodiments, a cochlear implant system signal processor (e.g., 120) can receive input signals from a middle ear sensor (e.g., 110) and/or other input source(s) and output signals to an electrical stimulator (e.g., 130) for stimulating the cochlear electrode (e.g., 116). In some embodiments, the same input signal can serve as a body parameter signal representative of an acoustic body parameter, for example, wherein a sound generated by the body (e.g., snoring, etc.) causes the input source to provide an input signal that represents such sound. Additionally or alternatively, an additional sensor, such as an implanted microphone, can provide such a body parameter signal representing one or more body sounds.

In some examples, a body sensor configured to output a body parameter signal representative of a body parameter can include a sensor configured to output a signal representative of one or more body sounds. In some such examples, the body sensor comprises an implanted microphone and/or a middle ear sensor. In some embodiments, such a body sensor provides input signals used for hearing perception. For instance, in some examples, such input signal can be received by the processor and the processor generates the stimulation signal based on the received input signals and a transfer function to generate stimulation signals.

Various possible body parameter measurements are possible and such measurements can be used in a variety of ways. In an example embodiment, a body sensor can include an electroencephalogram (EEG) sensor, which can be used, for example, to determine sleep stages of a wearer, such as if a wearer is in a REM sleep stage. Additionally or alternatively, other sensors can be used to measure or detect sleep, such as a heart rate monitor or an impedance sensor to detect changes in, for example, muscle activity.

In some examples, a body sensor can be configured to measure an electrical impedance within the body. As mentioned, changes in impedance between various locations within the body can indicate changes in muscle activity, which may provide information related to wearer sleep. In some examples, an electrode can be positioned on the costal margin of a wearer, and another electrode can be positioned in a pectoral region, such as proximate or as a part of a pectorally-implanted implantable battery and/or communication module, and impedance between the two electrodes can be measured to determine a body impedance between such electrodes.

In some such examples, another electrode can be positioned within the head of the wearer (e.g., an electrode as a part of a cochlear electrode, a signal processor and/or stimulator housing, or separate), and an electrical impedance between the head and the pectoral region (e.g., via an electrode near or on the battery) can be measured. The same or a different electrode in the pectoral region can be used when measuring impedance between the pectoral region and the head and between the pectoral region and the costal margin. In some example, an impedance can be measured between an electrode on an external surface of the implantable battery and/or communication module and a contact electrode or return electrode associated with cochlear stimulation. In some embodiments, the impedance between the head and the pectoral region can provide respiratory information regarding the wearer, such as inspiratory effort, inspiratory onset, inspiratory offset, paradoxical breathing, and/or variability of inspiratory duration. The system can be configured to output information (e.g., via an external device and/or cochlear stimulation) regarding a level of inspiratory effort.

In some examples, impedance values over time can be analyzed. For instance, timing of various impedance measurements can provide insight into inspiratory onset and offset. In some cases, inspiratory effort can include information regarding a magnitude of effort in combination with timing.

In some embodiments, impedance measurements can be broken down into real and imaginary components. Separating such components can allow identification of, for example, diaphragm movement/contraction compared to other muscles, such as intercostals. Differentiating between movement/contraction of such muscle groups can be valuable in detecting paradoxical breathing. Paradoxical breathing can be used as an indicator of obstructive sleep apnea.

In an example embodiment, a left electrode can be placed on a left side of a wearer's torso (e.g., to the left of the costal margin and below the armpit) and a right electrode can be placed on a right side of a wearer's torso (e.g., to the right of the costal margin and below the armpit). Impedance between the left and right electrodes can include an impedance associated with tissue including the wearer's diaphragm.

The diaphragm contracts and expands during breathing and the muscle changes shape. The changing shape of the diaphragm changes the capacitive properties of the tissue, affecting the impedance between the left and right electrodes. In particular, changing capacitance of the tissue between the left and right electrodes changes the imaginary part of the impedance between such electrodes.

The real component of the impedance across the chest, for example, the real component of the impedance between the left and right electrodes, provides information regarding broad chest activity, such as caused by activity of intercostal muscles. Other impedance measurements representing a different impedance vector (e.g., between a pectoral electrode and a costal margin electrode or one of the left or right electrodes) also can provide an indication of the broad chest activity.

During normal breathing, the intercostal muscles and diaphragm work together, and changes in the imaginary component of an impedance affected by the diaphragm (e.g., between the left and right electrodes referenced above) should occur along with changes in the real component between the left and right electrodes or other impedance vector during breathing. However, during paradoxical breathing, the diaphragm overpowers and the rib cage collapses compared to normal breathing patterns. Such action can affect impedance measurements regarding broad chest activity (e.g., intercostal activity) so that typical changes in the impedance (e.g., the real component of the impedance between left and right electrodes and/or another impedance vector) that would occur, e.g., due to intercostal activity, does not occur as expected.

Accordingly, in some examples, multiple impedance values can be used together to detect paradoxical breathing. For instance, in an example embodiment, a first impedance and a second impedance can be compared over time to detect paradoxical breathing. In some such examples, the first impedance measurement is the imaginary component of an impedance measured between left and right electrodes on opposite sides of the costal margin such as described above, and the second impedance measurement includes either the real component of the impedance measured between the left and right electrodes or a measurement of a different abdominal thoracic impedance vector. The first and second impedances can be analyzed over time. Changes in the first impedance can indicate activity of the diaphragm while changes in the second impedance can indicate activity of, e.g., the intercostal muscles. However, changes in only the first impedance without changes in the second impedance can indicate that the diaphragm is overpowering and the chest is collapsing, signaling paradoxical breathing.

In some examples, the first impedance and the second impedance can be analyzed together to search for paradoxical breathing. In some embodiments, a difference and/or a ratio of the magnitudes of the first and second impedance can be analyzed over time. The difference and/or ratio deviating from an expected value (e.g., due to the first impedance changing while the second does not) may indicate paradoxical breathing. In some examples, a comparison value (e.g., a ratio or a difference in magnitudes of the first impedance and the second impedance) satisfying a predetermined condition (e.g., falling outside of a predetermined range) implies paradoxical breathing.

Impedance values can also provide information regarding an activity level of a wearer. For instance, physical activity tends to make impedance between a wearer's chest and head more erratic. Impedance measured over time can provide information representative of how active a wearer over an observed time period, such as per 24-hour period. Such information can be provided, for example, to a wearer via an external device in communication with the implanted system to assist the wearer in tracking overall activity level. Tracked activity level can be used as a general health metric. In some embodiments, activity level can be used to assess a sleep state of a wearer. For instance, in some embodiments, a wearer can silence a cochlear implant system prior to sleeping, for example, to disable or reduce electrical stimulation in response to acoustic signals received at an input source (e.g., a middle ear sensor). However, in some such examples, even while the implant system is silenced or turned down for sleep, one or more body sensors can be used to monitor an activity level of the wearer. The cochlear implant system can be configured to (e.g., via the signal processor), if information from such one or more body sensors detects activity suggesting the wearer is awake and active, re-enable operation of the system to provide electrical stimulation in response to detected acoustic signals or increase stimulation to from a turned-down state to typical operating levels.

Electrical impedances between electrodes can be measured, for example, by providing one of a current or a voltage between two electrodes and measuring the other of the current and the voltage between the two electrodes, and calculating the impedance via Ohm's law. In some embodiments, three-wire or four-wire impedance measurements can be performed. For example, in some embodiments, current is provided to flow between a first electrode and a second electrode, and a voltage is measured between a third electrode and a fourth electrode, separate from the first and second electrodes. Such current and voltage values can be used to determine an impedance value.

In some examples, an impedance between the pectoral region and the head and an impedance between the pectoral region and the costal margin can be measured over time and at approximately the same time. Such impedance information can be analyzed together to detect whether aspects of respiration are out of sync, to analyze inspiratory effort, and/or other metrics. Such measurements can be used to detect, for example, paradoxical breathing, sleep apnea, Cheyne-Stokes breathing, or other occurrences.

In some examples, impedance measurements can be used to detect paradoxical breathing, as discussed elsewhere herein. Detection of paradoxical breathing can indicate obstructive sleep apnea events are occurring. If those events occur often enough then it indicates the person may have obstructive sleep apnea.

Detection of presence or absence of inspiratory effort allows determination if central apneas are occurring. Detection of magnitude of inspiratory effort is helpful to determine if Cheyne-Stokes breathing is occurring because Cheyne-Stokes has distinctive recurring alternating periods of crescendo-decrescendo breathing separated by central apneas.

Accordingly, various data from one or more body sensors can be used to detect various conditions, such as sleep apnea and Cheyne-Stokes breathing. In various examples, one or more devices (e.g., an implanted signal processor in communication with one or more body sensors, an external device receiving body parameter information, or the like) can be programmed to recognize one or more conditions, such as sleep apnea, Cheyne-Stokes breathing, or others based on data from implanted body sensors.

A system component receiving and analyzing body parameter information (e.g., a signal processor of an implanted cochlear implant system, an external device) can be configured to analyze such signals and, if one or more conditions (e.g., Cheyne-Stokes breathing, sleep apnea, paradoxical breathing) are detected, output an alert indicating such (e.g., via a user interface of an external device or cochlear stimulation).

Additionally, detected Cheyne-Stokes breathing can be analyzed in combination with additional contemporaneous body parameter signals, such as an EEG signal. In some examples, an EEG signal can be used to determine a sleep stage of a wearer. In some cases, out-of-sync impedances such as those indicating Cheyne-Stokes breathing can also occur during REM sleep. An EEG signal can be used to confirm whether out-of-sync impedance values coincide with REM sleep, where such values may be expected, or not, which may indicate Cheyne-Stokes breathing.

In another example, as described herein, in some embodiments, a body sensor can provide information regarding a body orientation of the wearer. The position of such a sensor (e.g., an accelerometer) can provide information regarding the orientation of the body part where the sensor is located. Multiple such sensors can provide information regarding relative orientation between multiple associated body parts. In some examples, orientation information can be used by a system to determine desired cochlear stimulation. In an example embodiment, an accelerometer is positioned within a wearer's head and can provide information regarding whether the wearer's head is tilted back, tucked downward, or looking forward. A cochlear implant system can be configured to provide different levels of electrical stimulation to cochlear tissue based on the orientation.

For instance, in some examples, a wearer can disable or turn down electrical stimulation based signals from an input source, for example, for sleeping. In an example configuration, a signal processor can be configured to detect that a wearer is vertical, suggesting the person is awake and active and re-enable stimulation or restore stimulation magnitude to typical operating levels based on the orientation information.

In some examples, orientation-based aspects of stimulation can be tested on a user-by-user basis to determine how orientation affects an efficacy of stimulation. In some examples, a signal processor can be configured to generate a stimulation based on a received input signal, received body parameter information representing orientation information, and a signal processor transfer function.

In still a further embodiment, an accelerometer can provide information regarding external impacts on the body proximate the accelerometer. For example, an accelerometer in a chest area of a wearer can provide information regarding external impacts on the chest. Such an accelerometer can be used as an interface with an implanted cochlear system, wherein accelerometer information can sense an intentional impact initiated by a user (e.g., a wearer tapping on an area of the chest proximate an accelerometer). A detected impact can function as an input to the system, wherein the system is configured to respond according to the detected impact. In an example, a detected impact proximate an accelerometer (e.g., in the chest area) acts as a quick shut-off of a cochlear implant system. Various functions can be associated with such a sensor. A cochlear implant system can be programmed such that the signal processor responds in a predetermined way in response to a signal received from such an implanted accelerometer detecting an impact.

As described herein, one or more body sensors can be used to observe one or more body parameters, such as by presenting body parameter information to a wearer or a clinician. In various examples, body parameter information can be presented via direct stimulation, such as via electrical stimulation of cochlear tissue, and/or via external presentation, such as via one or more speakers and/or a visual display. In various examples, body parameter information can be presented one or more ways in response to a received command requesting body parameter information and/or automatically in response to the body parameter meeting a predetermined threshold condition.

Various non-limiting embodiments have been described. These and others are within the scope of the following claims.

1. A system comprising:
an implantable body sensor configured to measure a first body parameter and generate a first body parameter signal representative of the first body parameter; and
a cochlear implant system in communication with the implantable body sensor, the cochlear implant system configured to:
provide electrical stimulation to cochlear tissue in response to one or more input signals; and
receive the first body parameter signal from the implantable body sensor.

2. The system of claim 1, wherein the cochlear implant system is configured to provide electrical stimulation to the cochlear tissue based on the received first body parameter signal and representative of the first body parameter such that a wearer perceives a representation of the first body parameter.

3. The system of claim 1, wherein the cochlear implant system comprises:
an input source configured to receive a stimulus and generate an input signal representative of the stimulus;
a cochlear electrode;
a stimulator in communication with the cochlear electrode and configured to provide the electrical stimulation to the cochlear tissue via the cochlear electrode;
a signal processor in communication with the stimulator and the input source, the signal processor being programmed with a transfer function and being configured to receive the one or more input signals from the input source and output a stimulation signal to the stimulator based on the received one or more input signals and the transfer function; and
an implantable battery and/or communication module in communication with the signal processor and the implantable body sensor, the implantable battery and/or communication module configured to provide electrical power to the signal processor and receive the first body parameter signal from the implantable body sensor.

4. The system of claim 3, wherein
the implantable battery and/or communication module is configured to output body parameter information based on the received first body parameter signal to the signal processor; and
the signal processor is configured update the transfer function based on the received body parameter information.

5. The system of claim 4, wherein the body parameter information comprises the first body parameter signal such that the implantable battery and/or communication module is configured to output the first body parameter signal to the signal processor.

6. The system of claim 1, wherein:
the cochlear implant system is configured to determine if the first body parameter is outside of a predetermined range; and
if the first body parameter is outside the predetermined range, the cochlear implant system is configured to output an alert signal.

7. The system of claim 6, wherein the cochlear implant system is configured to output the alert signal to an external device.

8. The system of claim 6, wherein outputting the alert signal comprises outputting electrical stimulation.

9. The system of claim 1, wherein the cochlear implant system is configured to output body parameter information representative of the first body parameter to an external device.

10. The system of claim 1, wherein the implantable body sensor is configured to be implanted proximate a wearer's chest.

11. The system of claim 1, further comprising a second implantable body sensor configured to measure a second body parameter, the second body parameter being different from the first, and output a second body parameter signal representative of the second body parameter; and wherein the cochlear implant system is configured to receive the second body parameter signal.

12. The system of claim 11, wherein the implantable body sensor and the second implantable body sensor are housed in a single housing.

13. The system of claim 11, wherein the cochlear implant system is configured to determine if the second body parameter is outside of a predetermined range; and
if the second body parameter is outside the predetermined range, the cochlear implant system is configured to output an alert signal.

14. The system of claim 1, wherein the first body parameter comprises heart rate, body temperature, an electrocardiogram, electroencephalogram (EEG), blood oxygen levels, glucose levels, inspiratory effort levels, body orientation, body impedance measurements, wearer activity level, or sleep stage information.

15. A cochlear implant system comprising:
a cochlear electrode;
a stimulator in electrical communication with the cochlear electrode;
an implantable body sensor configured to measure a body parameter of a wearer and generate a body parameter signal based on the measured body parameter;
a signal processor in communication with the stimulator and the implantable body sensor, the signal processor being programmed with a transfer function and being configured to:
receive the body parameter signal; and
output a stimulation signal to the stimulator based on the transfer function.

16. The cochlear implant system of claim 15, wherein the signal processor is further configured to output the stimulation signal based on the body parameter signal.

17. The cochlear implant system of claim 15, further comprising an implantable battery and/or communication module in communication with the implantable body sensor and the signal processor, and wherein the implantable battery and/or communication module is configured to:
provide electrical power to the signal processor;
receive the body parameter signal from the implantable body sensor; and
communicate the received body parameter signal to the signal processor such that the signal processor receives the body parameter signal from the implantable battery and/or communication module.

18. The cochlear implant system of claim 17, wherein the signal processor is configured to:
determine the body parameter based on the received body parameter signal;
compare the body parameter to a predetermined range; and
if the body parameter is outside of the predetermined range, output an alert.

19. The cochlear implant system of claim 18, wherein:
outputting the alert comprises outputting an alert signal to the implantable battery and/or communication module indicating that the body parameter is outside of the predetermined range; and
the implantable battery and/or communication module is configured to wirelessly output an alert to an external device.

20. The cochlear implant system of claim 18, wherein outputting the alert comprises generating and outputting an alert stimulation signal to the stimulator, the alert stimulation signal providing information regarding the body parameter.
